(19) United States
(12) Patent Application Publication
(10) Pub. No.: US 2020/0197596 A1
(43) Pub. Date: Jun. 25, 2020

(54) DIALYSIS CATHETER

(71) Applicant: Clifford Okundaye, Rapid City, SD (US)

(72) Inventor: Clifford Okundaye, Rapid City, SD (US)

(21) Appl. No.: 16/644,410

(22) PCT Filed: Sep. 14, 2018
(86) PCT No.: PCT/US2018/051130

§ 371 Date: Mar. 4, 2020

Related U.S. Application Data
(63) Continuation-in-part of application No. 15/704,272, filed on Sep. 14, 2017.

(51) Int. Cl. A61M 1/36; A61M 25/01; A61M 25/00; A61M 25/06

(52) U.S. Cl. CPC A61M 1/3661; A61M 25/0194; A61M 25/0028; A61M 25/0029; A61M 2025/0286; A61M 25/0082; A61M 25/0668; A61M 2210/125; A61M 25/0097

(57) ABSTRACT

A dialysis catheter includes an entirely subcutaneous first section, with an exterior interface via penetration of the overlying subcutaneous skin layer. In addition the first section of the dialysis catheter includes a detachable portion that enables insertion via a guidewire instead of a peel-away sheath. The dialysis catheter has a layered design that minimizes risk of infection.

### CROSS-REFERENCE TO RELATED APPLICATIONS

This application is a filing under 35 U.S.C. 371 of International Application No. PCT/US2018/051130 filed Sep. 14, 2018, which claims priority to and is a continuation-in-part of U.S. patent application Ser. No. 15/704,272 filed Sep. 14, 2017 by Clifford Okundaye and entitled “Dialysis Catheter” which is incorporated herein by reference as if reproduced in its entirety. For purposes of nationalization in the United States, the present application is a continuation-in-part of U.S. patent application Ser. No. 15/704,272 filed Sep. 14, 2017.

### FIELD OF THE INVENTION

The present invention is related to dialysis catheters as a means of accessing blood from the body for filtering by a dialysis machine.

### BACKGROUND

Catheters have been in existence for various purposes, including draining of abscess and for urinary issues, since the 19th century. Dialysis involve the process of cleaning toxins present in large quantities in patients' blood through a filter membrane, using a pump process, and then returning the clean or dialyzed blood back to the patient. In the 1960s dialysis was discovered as a tool of improving survival in patients with end stage kidney disease or kidney failure. The patient blood is obtained from a large central vein via a dialysis catheter, which includes tubes that allow blood to be drawn from the patient, the blood then being processed through a dialysis machine and returned back to the patient through a different part of the tubes or dialysis access.

FIG. 1 illustrates a conventional in vitro dialysis catheter 100 in vitro, in accordance with one embodiment. The dialysis catheter 100 may comprise a lumen tube 112 having a distal end 102 and a proximal end 104. The proximal end 104 bifurcates into limbs 106, each of which includes one or more clamps 110. The limbs 106 terminate at limb ports 108. FIG. 2 illustrates a dialysis catheter insertion process 200 in accordance with one embodiment. To insert the dialysis catheter, first cannulate the central vein with a needle at an insertion site 202. Next, advance a guidewire into the central vein 204, and remove the needle 206. Next, tunnel from the insertion site to create a tunneled layer 208, and insert the dialysis catheter through the tunneled layer and over the guidewire 210. Then remove the guidewire 212 and insert sutures at an exit site of the dialysis catheter 214, and insert sutures at the insertion site 216.

In some cases, a peel away sheet is deployed over the guidewire to facilitate removal of the guidewire and placement of the tunneled dialysis catheter at the insertion site. This technique may require use of a flow valve. Next, align the dialysis catheter such that limb ports of the dialysis catheter are outside the tunneled layer 218, and deploy occluding clips on the limb ports 220, and anchor the limb ports to the skin 222.

Initially at the advent of dialysis, access to the patient blood was mainly via such tubes. Recently, AV grafts and AV fistulas have become the preferred method for accessing the blood, due to lower risk of infection. Dialysis catheters are still common in patients starting dialysis for the first time, and in many others who cannot tolerate a fistula or graft. The advantage of the dialysis catheter includes easy access and early use from the time of insertion to the time when dialysis is needed. A major disadvantage is the high risk of infection.

Studies have shown that in long term dialysis patients using a tunneled dialysis catheter, the exit site of the dialysis catheter infection is strongly correlated to the exit site bacteria contamination. Prevention of swimming or exposure to water has been tried to minimize recurrent infection or bacterial contamination of the exit site. However, the exposure of the catheter exit site to the skin results in a high chance of bacteria migrating to contaminate the dialysis catheter and subsequently cause bacteremia infections.

In the early 1990s, an entirely subcutaneous dialysis catheter called Life Site was introduced, but was not shown to decrease dialysis catheter related infections. The Life Site catheter utilized a complex valve system, involving multiple metallic and silicone layers inside the valves.

### SUMMARY

In an embodiment, a dialysis catheter may comprise a lumen tube comprising an inflow lumen and an outflow lumen; a clampless first limb leading to the inflow lumen; a clampless second limb leading to the outflow lumen; a plurality of limb ports attached to the first limb and the second limb, the limb ports comprising: a fitting configured to attach to the first or second limb; a needle guide configured to attach to the fitting, and configured to guide a needle into the first or second limb; and at least one valve positioned between the needle guide and the fitting, configured to prevent fluid flow in one direction.

In an embodiment, a method of inserting a dialysis catheter into a patient may comprise inserting a trocar in the patient in a first direction at an incision site; using the trocar to create a subcutaneous tissue tunnel from the incision site to an exit site from the patient; inserting the trocar at the exit site in a second direction different from the first direction; using the trocar to create a tissue tunnel extension without exiting the patient again; inserting the dialysis catheter into the exit site; extending the dialysis catheter from the exit site through a central vein or jugular vein into a superior vena cava; configuring two limbs of the dialysis catheter each with a limb port housing a valve, a needle guide coupled to the valve, and a cover on the needle guide; and inserting the limbs of the dialysis catheter into the subcutaneous tissue tunnel and the tissue tunnel extension.

In an embodiment, a dialysis catheter may comprise a lumen tube bifurcating into a first limb and a second limb; a plurality of limb ports attached to the first limb and the second limb, the limb ports comprising a fitting configured to attach to the first or second limb, and configured to house a valve; and a needle guide configured to attach to the fitting, and configured to retain the valve intermedially between the needle guide and the limb port.

### BRIEF DESCRIPTION OF THE DRAWINGS

To easily identify the discussion of any particular element or act, the most significant digit or digits in a reference number refer to the figure number in which that element is first introduced.

FIG. 1 illustrates a conventional in vitro dialysis catheter 100 in accordance with one embodiment.

FIG. 2 illustrates a conventional dialysis catheter insertion process 200.

FIG. 3 illustrates a perspective view of a dialysis catheter 300 in accordance with one embodiment.

FIG. 4 illustrates a cutaway view of a dialysis catheter 300 in accordance with one embodiment.

FIGS. 5A-5C illustrate a fitting 310 in accordance with one embodiment.

FIGS. 6A-6D illustrate a valve 308 in accordance with one embodiment.

FIGS. 7A-7D illustrate a needle guide 312 in accordance with one embodiment.

FIGS. 8A-8C illustrate a limb port assembly 301 in accordance with one embodiment.

FIG. 9 illustrates a dialysis catheter insertion process 900 in accordance with one embodiment.

FIG. 10 illustrates a dialysis catheter insertion process 1000 in accordance with one embodiment.

FIG. 11 illustrates an in vitro dialysis catheter 1100 in accordance with one embodiment.

FIGS. 12A-12D illustrate an arterial port assembly 1200 in accordance with one embodiment.

FIGS. 13A-13D illustrate a venous port assembly 1300 in accordance with one embodiment.

FIGS. 14A-14D illustrate a needle guide 1400 in accordance with one embodiment.

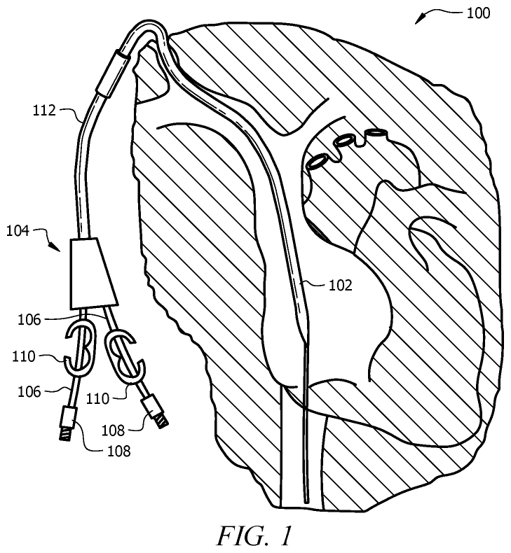

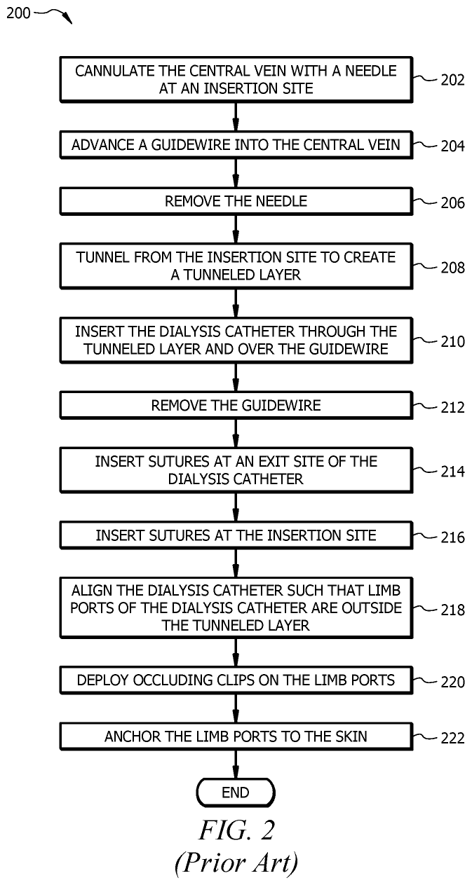

### DETAILED DESCRIPTION

Various terms are used herein and should be accorded their conventional meaning in the relevant arts, unless otherwise defined expressly or by context.

“Cannulate” herein refers to insertion of a tubular structure into a vein or body cavity.

“Exit site” herein refers to the site where the proximal portion of the catheter exits the subcutaneous surface for performing dialysis.

“Luminal flow” herein refers to the flow of blood through tubular structures of the dialysis catheter.

The high cost of treatment and the high mortality and morbidity rate of catheter related bacteremia creates an ongoing need for a dialysis catheter that is entirely subcutaneous when not in use, and that minimizes exposure to the overlying skin layer, thereby preventing exit sites from being a source of bacteremia.

A tunneled dialysis catheter is disclosed that is entirely subcutaneous after insertion and which does not utilize an opening in the skin for catheter exit when not in use, thereby minimizing risk of transmission of bacteria via the tunneled exit site from the skin surface. The tunneled dialysis catheter utilizes a minimal valve system and enables high luminal flow to minimize the need for antibiotics.

Additionally, a device as described herein may be used in other areas, for example by adjusting the sizes of the various proportions of the elements of the limb port. As an example, the limb port embodiments described herein could be used as a modified subcutaneous intraabdominal device for drainage of recurrent ascites. As another example, the limb port embodiments described herein could be used as a modified alternative device for drainage of recalcitrant pleural effusions. As another example, the limb port embodiments described herein could be used as a subcutaneous drainage catheter for cerebrospinal fluid collection and drug injection. As another example, the limb port embodiments described herein could be used as a modified device for percutaneous bladder drainage.

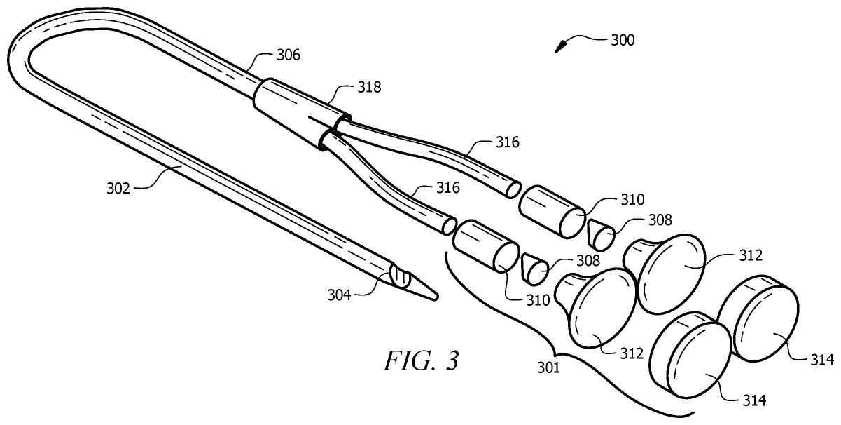

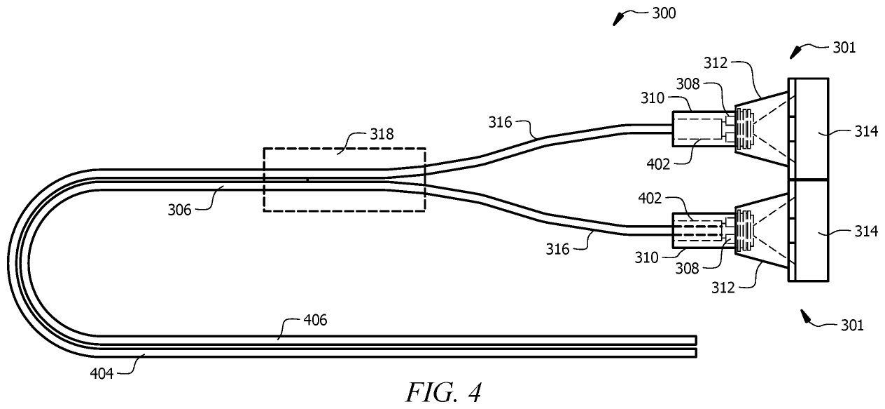

FIG. 3 and FIG. 4 illustrate a dialysis catheter 300 in accordance with one embodiment. The dialysis catheter 300 may comprise a catheter lumen tube 302 having a distal end 304 and a proximal end 306. In some embodiments, the catheter lumen tube 302 may comprise an inflow lumen 404 (for inflow toward the patient's heart) and an outflow lumen 406 (for outflow away from the patient's heart). In other words, the inflow lumen 404 may also be called the venous lumen and the outflow lumen 406 may also be called the arterial lumen. Although both lumens are in the vein of the patient, the “arterial” lumen, like natural arteries, carries blood away from the heart, while the “venous” lumen returns blood towards the heart.

The proximal end 306 of the lumen tube 302 may comprise (and/or be attached to) at least two limbs 316. The limbs 316 at the proximal end 306 may be joined into the catheter lumen tube 302 by a junction housing 318. Each of the limbs 316 may be clampless (i.e., function without the use of clamps to prevent fluid from flowing out of the limbs 316).

Each of the limbs 316 may be terminated by a limb port (or limb port assembly) 301. The limb port 301 may comprise a fitting 310 including an opening or through-hole 402, into which one or more valve 308 (e.g., a duckbill valve, a mini valve) may be disposed, followed by a needle guide 312 and cover 314. In some embodiments, one or more elements of the limb port 301 (such as the fitting 310 and the needle guide 312 for example) may comprise stainless steel, titanium, plastic, and/or any other material with an appropriate shore hardness.

The clampless design of the limbs 316 may be due to the function of the limb port 301, and particularly the valve 308 within the limb port 301. The valve 308 may comprise a one-way valve that allows fluid to flow into the limbs 316 and subsequently the limen tube 302 while preventing fluid from flowing out of the limb port 301. In some embodiments, a plurality of valves 308 may be positioned within the fitting 310 to safeguard against failure of one of the valves 308.

In some embodiments, the cover 314 may allow a needle to puncture the material of the cover 314 and pass into the needle guide 312 and then possibly the valve 308, and the material of the cover 314 may seal with the needle around the puncture to prevent fluid flow out of the cover 314. In some embodiments, the cover 314 may comprise an expandable material such as silicone and/or expanded Polytetrafluoroethylene (ePTFE). The valve 308 may allow fluid to flow from a needle into the fitting 310 and/or limb(s) 316 of the lumen tube 302. The clampless design may allow for the catheter 300 to be positioned approximately entirely under a patient's skin. For example, the limb port 301 may be accessed while at least a majority of the catheter 300 is positioned under a patient's skin.

In one embodiment, the valve 308 may be retained freely (without adhesive, clips, clamps etc.) between the needle guide 312 and the fitting 310. Thus, unscrewing or otherwise detaching the needle guide 312 from the fitting 310 will free the valve 308 for removal and cleaning or replacement, for example.

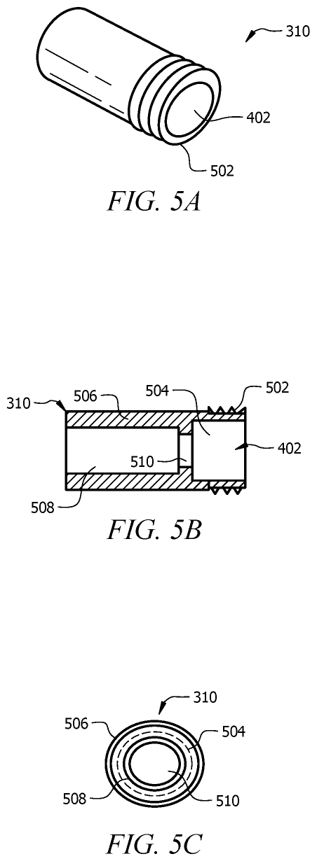

FIGS. 5A-5C illustrate a fitting 310 of a limb port in accordance with one embodiment. The fitting 310 may comprise threads 502 configured to interface with threads of one or more other elements of a limb port. The fitting 310 may comprise a valve chamber 504 (which may be built into or shaped into the through-hole 402) configured to receive and/or fit around a valve of a limb port. The fitting 310 may comprise a casing 506 (or housing) forming the outer shell/surface of the fitting 310 and surrounding the through-hole 402. The fitting 310 may comprise a limb dock 508 (which may be built into or shaped into the through-hole 402) configured to receive and/or fit around a limb of a catheter lumen tube. The fitting 310 may comprise a transition region 510 between the limb dock 508 and the valve chamber 504 (which may be built into or shaped into the through-hole 402), wherein the transition region 510 may provide a space between the valve (within the valve chamber 504) and the limb (within the limb dock 508).

Each of the limbs 316 (described above) may be inserted and secured into the limb dock 508 of the respective fitting 310, and for each fitting a valve 308 (described above) may be disposed within the valve chamber 504. In the illustrated embodiment, the needle guide 312 (described above) may be secured to the fitting 310 by screwing it over the threads 502.

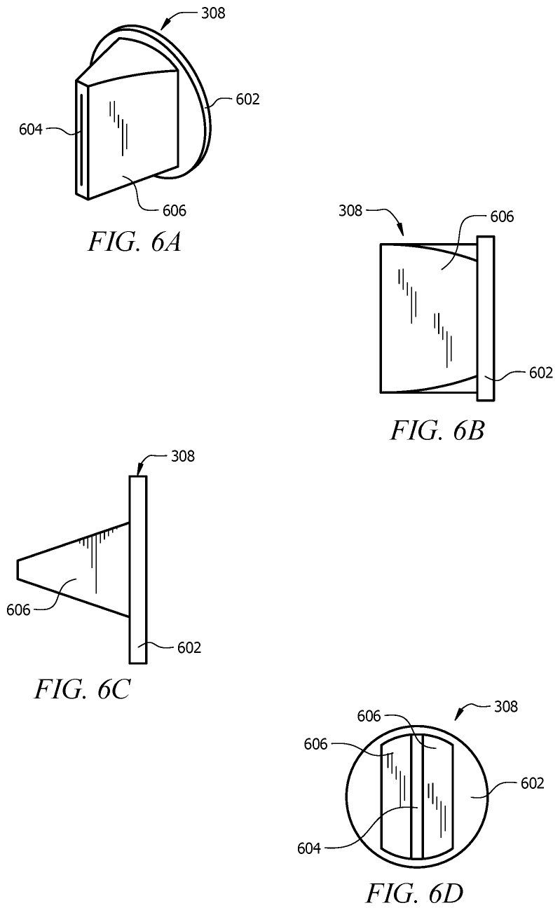

FIGS. 6A-6D illustrate a valve 308 in accordance with one embodiment. The valve 308 may be configured to fit within the valve chamber of the fitting. The valve 308 may comprise a stop cover 602, a valve port 604, and a port body 606.

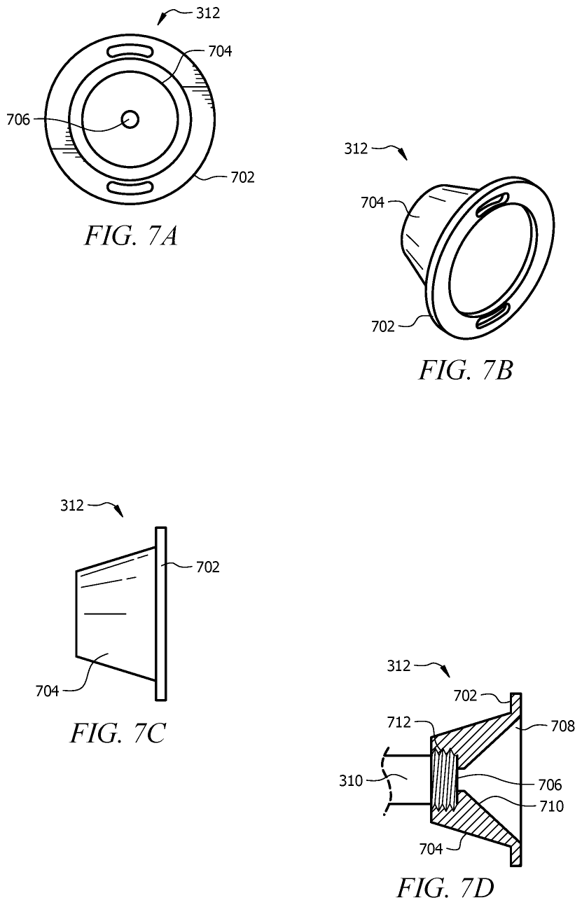

FIGS. 7A-7D illustrate a needle guide 312 in accordance with one embodiment. The needle guide 312 may comprise a flange 702 configured to allow the needle guide 312 to attach to the cover (described above). The needle guide 312 may comprise a tapered body 704 configured to direct a needle through the needle guide 312. The needle guide 312 may comprise a needle aperture 706 configured to allow a needle to exit the needle guide 312 at a desired position. The needle guide 312 may comprise internal threads 712 to mate with the threads 502 of the fitting 310 (described above). The tapered body 704 may comprise a tapered interior that tapers from a wide aperture 708 to a narrow aperture 710 at the interface to the valve 308. The narrow aperture 710 may direct a needle toward the needle aperture 706, which may be positioned within and/or proximately to the threads 712.

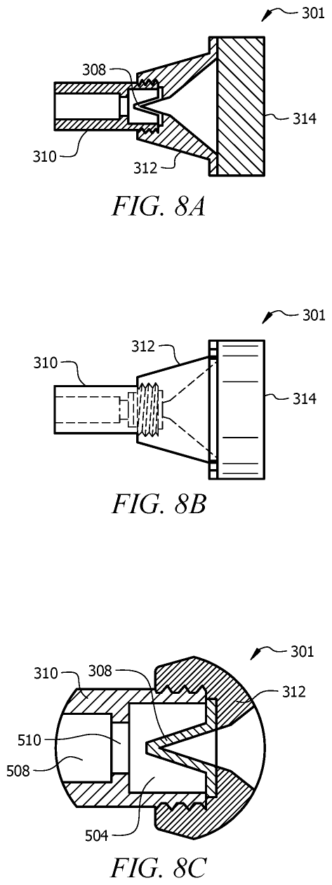

FIGS. 8A-8C illustrates a limb port assembly 301 in accordance with one embodiment. The limb port assembly 301 may be formed by inserting the valve 308 into the through-hole 402 of the fitting 310, the needle guide 312 may be rotated onto the threads 502 of the fitting 310, and the cover 314 may be pressure fit to the flange 702 of the needle guide 312. In some embodiments, a plurality of valves 308 may be positioned within the fitting 310 (i.e., between the needle guide 312 and the limb dock 508).

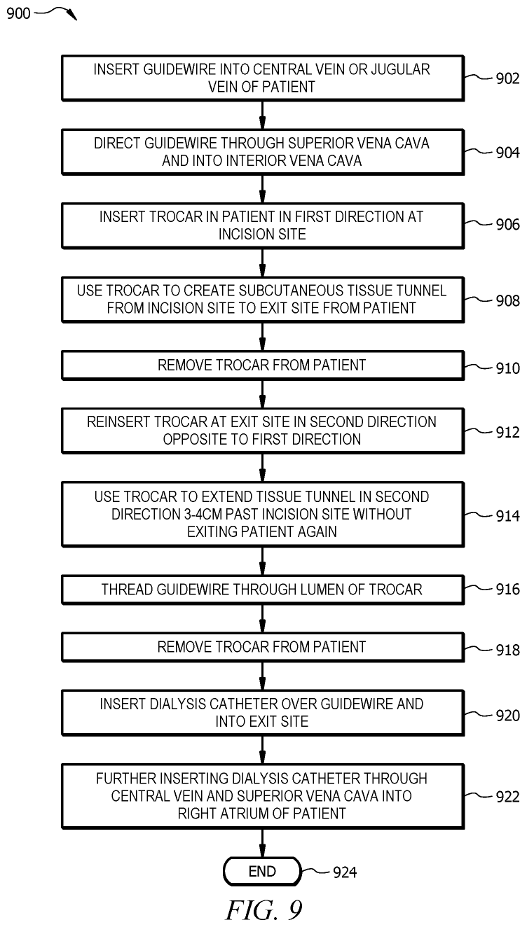

FIG. 9 illustrates a dialysis catheter insertion process 900 in accordance with one embodiment. In block 902 of the process 900, the medical provider inserts a guidewire into a central vein or jugular vein of the patient. In block 904, the guidewire is directed through the superior vena cava and into the interior vena cava. In block 906, a trocar is inserted into the patient in a first direction at an incision site.

In block 908, the trocar is used to create a subcutaneous tissue tunnel from the incision site to an exit site from the patient. In block 910, the trocar is removed from the patient. The subcutaneous tissue tunnel is then modified as follows.

In block 912, the trocar is inserted at the exit site in a second direction different from the first direction. In block 914, the trocar is used to extend the tissue tunnel in the second direction approximately 3 to 4 centimeters without exiting the patient again. The patient is now prepared for insertion of the guidewire.

In block 916, the guidewire is threaded through a lumen of the trocar. In block 918, the trocar is removed from the patient, in preparation for insertion of the dialysis catheter.

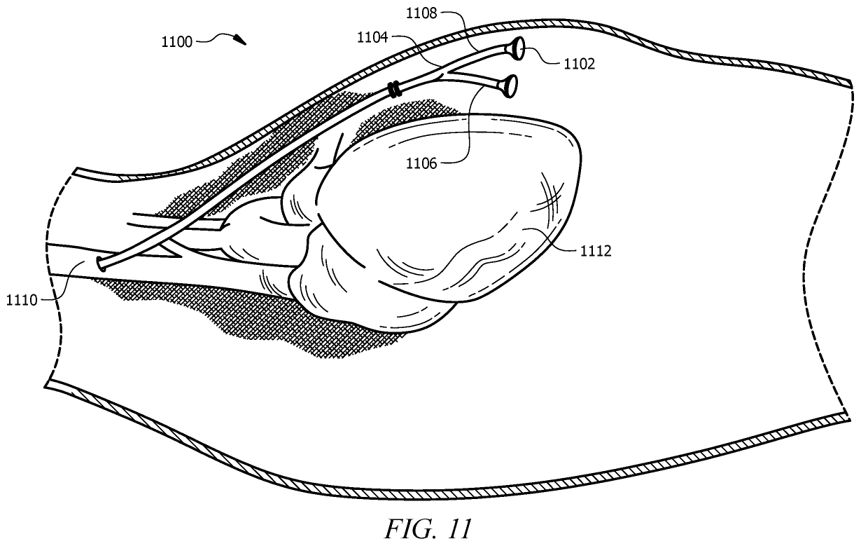

In block 920, the dialysis catheter is inserted over the guidewire and into the exit site. In block 922, the dialysis catheter is further inserted through the central vein and superior vena cava into a right atrium of the patient, and the limbs are inserted into the subcutaneous tissue tunnel and the tissue tunnel extension (see FIG. 11). Upon completion at block 924, the dialysis catheter insertion process 900 ends.

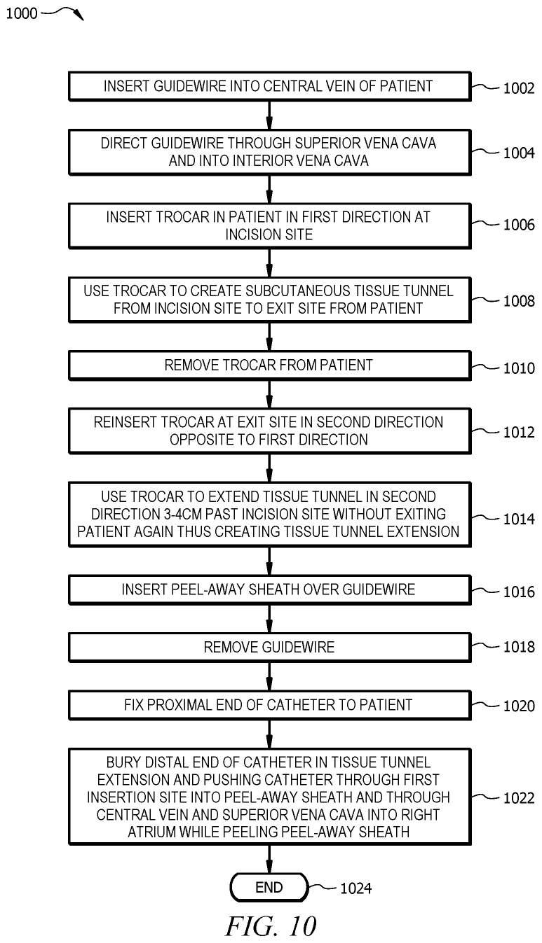

FIG. 10 illustrates a dialysis catheter insertion process 1000 in accordance with another embodiment. At block 1002, the medical provider inserts a guidewire into a central vein of the patient. In block 1004, the guidewire is directed through the superior vena cava and into the interior vena cava. In block 1006, a trocar is inserted into the patient in a first direction at an incision site. In block 1008, the trocar is used to create a subcutaneous tissue tunnel from the incision site to an exit site from the patient. Blocks 1002 through 1008 are similar to those described in the process 900 of FIG. 9.

In block 1010, the trocar is removed from the patient. In block 1012, the trocar is reinserted at the exit site in a second direction different from the first direction. In block 1014, the trocar is used to extend the tissue tunnel in the second direction approximately 3 to 4 centimeters without exiting the patient again thus creating a tissue tunnel extension. At this point, the process 1000 diverges somewhat from the process 900 in FIG. 9.

In block 1016, a peel-away sheath is inserted over the guidewire. In block 1018, the guidewire is removed. In block 1020, a proximal end of the catheter is fixed to the patient. In block 1022, a distal end of the catheter is buried in the tissue tunnel extension and the catheter is pushed through the first insertion site into the peel-away sheath and through the central vein and superior vena cava into the right atrium, while peeling the peel-away sheath. Upon completion at block 1024, the process 1000 ends.

FIG. 11 illustrates an in vitro dialysis catheter 1100 in accordance with one embodiment. The in vitro dialysis catheter 1100 may be inserted at an incision site 1102 following, for example, a guidewire along a central vein to the superior vena cava 1110 and into the heart 1112. A trocar is utilized to form a tissue tunnel 1108 from an incision site 1102 to an exit site 1104 from which the dialysis catheter is accessed. The trocar is also utilized to form a tissue tunnel extension 1106. When not in use, both limbs of the dialysis catheter may be completely inserted (one in the tissue tunnel 1108, the other in the tissue tunnel extension 1106) in vitro and do not protrude from the body of the patient (as described above).

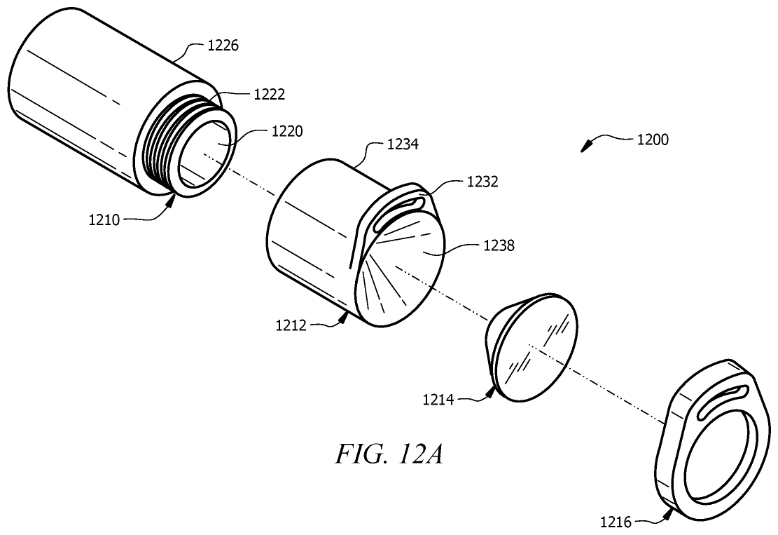

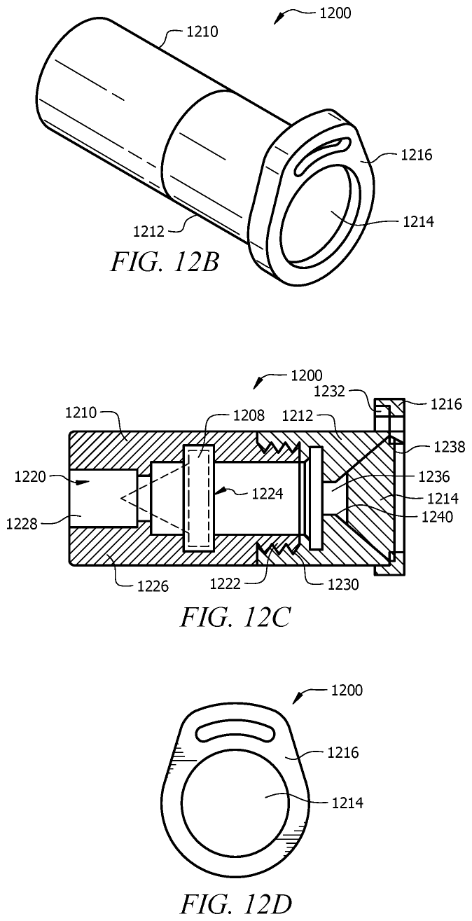

FIGS. 12A-12D illustrate an embodiment of a limb port assembly (as described above) that may comprise an arterial port assembly 1200. The arterial port assembly 1200 may be attached to an arterial lumen of the dialysis catheter. Although both lumens are in the vein, the “arterial” lumen, like natural arteries, carries blood away from the heart, while the “venous” lumen returns blood towards the heart. In other words, the arterial lumen may also be known as the outflow lumen 406 (described in FIG. 4) and the venous lumen may also be known as the inflow lumen 404.

The arterial port assembly 1200 may comprise a fitting 1210, a needle guide 1212, a silicone cover 1214 and a silicone cover retainer 1216. The silicone cover 1214 may be configured to fit within the needle guide 1212, and the silicone cover retainer 1216 may be configured to attach to the needle guide 1212 to retain the silicone cover 1214 within the needle guide 1212. The needle guide 1212 may comprise internal threads configured to engage with threads on the fitting 1210. In some embodiments, the silicone cover 1214 may comprise PTFE instead of silicone.

The fitting 1210 may comprise threads 1222 configured to interface with threads 1230 of the needle guide 1212. The fitting 1210 may comprise a valve chamber 1224 (which may be built into or shaped into the through-hole 1220) configured to receive and/or fit around a valve 1208 (shown in FIG. 12C). In some embodiments, a plurality of valves 1208 may be positioned within the fitting 1210. The fitting 1210 may comprise a casing 1226 (or housing) forming the outer shell/surface of the fitting 1210 and surrounding the through-hole 1220. The fitting 1210 may comprise a limb dock 1228 (which may be built into or shaped into the through-hole 1220) configured to receive and/or fit around a limb of a catheter lumen tube.

The needle guide 1212 may comprise a flange 1232 configured to allow the needle guide 1212 to attach to the cover 1214 and/or cover retainer 1216. The needle guide 1212 may comprise a tapered body 1234 configured to direct a needle through the needle guide 1212. The needle guide 1212 may comprise a needle aperture 1236 configured to allow a needle to exit the needle guide 1212 at a desired position. The needle guide 1212 may comprise internal threads 1230 to mate with the threads 1222 of the fitting 1210. The tapered body 1234 may comprise a tapered interior that tapers from a wide aperture 1238 to a narrow aperture 1240. The narrow aperture 1240 may direct a needle toward the needle aperture 1236, which may be positioned within and/or proximately to the threads 1230.

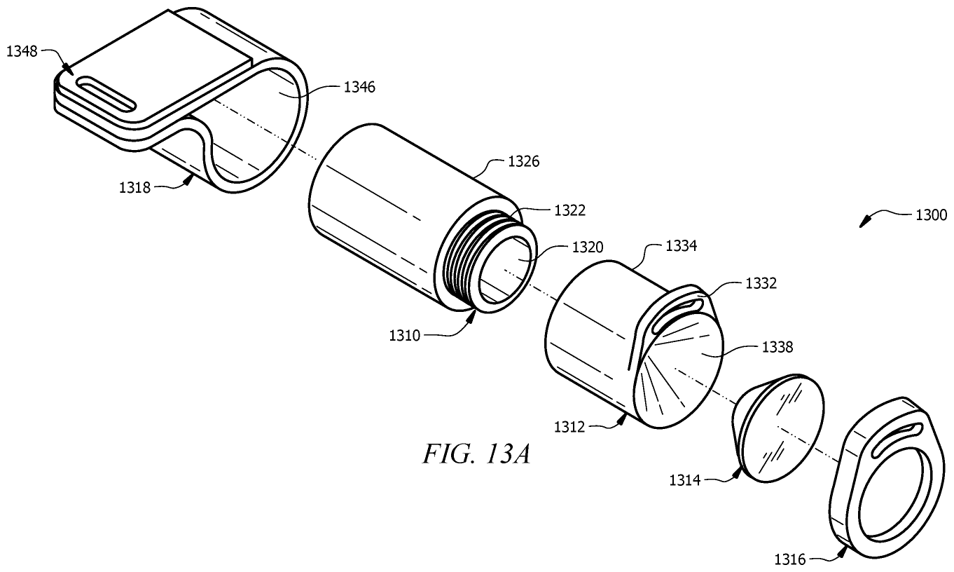

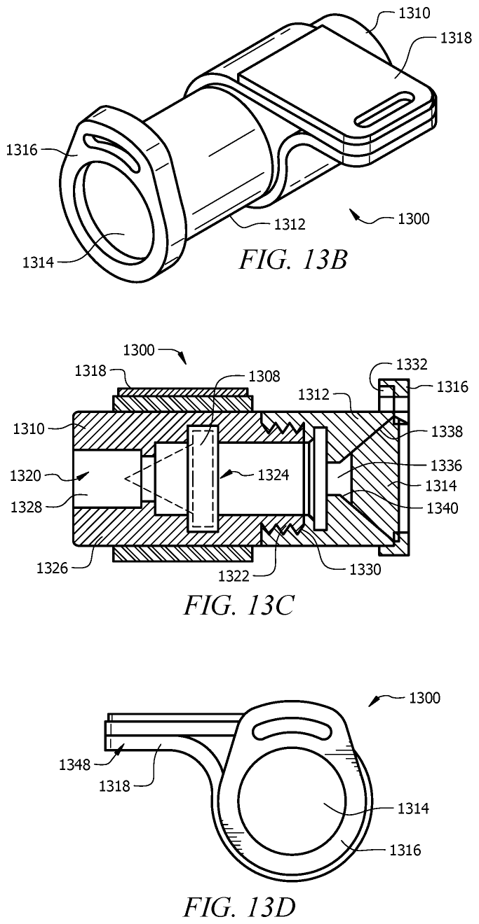

FIGS. 13A-13D illustrate an embodiment of a limb port assembly (as described above) that may comprise a venous port assembly 1300. The venous port assembly 1300 may be attached to a venous lumen of the dialysis catheter. The venous port assembly 1300 may comprise a fitting 1310, a needle guide 1312, a silicone cover 1314 and a silicone cover retainer 1316. In some embodiments, the venous port assembly 1300 may also comprise a suture flap 1318. The silicone cover 1314 may be configured to fit within the needle guide 1312, and the silicone cover retainer 1316 may be configured to attach to the needle guide 1312 to retain the silicone cover 1314 within the needle guide 1312. The needle guide 1312 may comprise internal threads configured to engage with threads on the fitting 1310.

The fitting 1310 may comprise threads 1322 configured to interface with threads 1330 of the needle guide 1312. The fitting 1310 may comprise a valve chamber 1324 (which may be built into or shaped into the through-hole 1320) configured to receive and/or fit around a valve 1308 (shown in FIG. 13C). In some embodiments, a plurality of valves 1308 may be positioned within the fitting 1310. The fitting 1310 may comprise a casing 1326 (or housing) forming the outer shell/surface of the fitting 1310 and surrounding the through-hole 1320. The fitting 1310 may comprise a limb dock 1328 (which may be built into or shaped into the through-hole 1320) configured to receive and/or fit around a limb of a catheter lumen tube.

The needle guide 1312 may comprise a flange 1332 configured to allow the needle guide 1312 to attach to the cover 1314 and/or cover retainer 1316. The needle guide 1312 may comprise a tapered body 1334 configured to direct a needle through the needle guide 1312. The needle guide 1312 may comprise a needle aperture 1336 configured to allow a needle to exit the needle guide 1312 at a desired position. The needle guide 1312 may comprise internal threads 1330 to mate with the threads 1322 of the fitting 1310. The tapered body 1334 may comprise a tapered interior that tapers from a wide aperture 1338 to a narrow aperture 1340. The narrow aperture 1340 may direct a needle toward the needle aperture 1336, which may be positioned within and/or proximately to the threads 1330.

The suture flap 1318 may comprise a through-hole 1346 configured to fit around the fitting 1310, and a flap portion 1348 configured to extend to allow for a suture to pass through the flap portion 1348 and secure the suture flap 1318 (and therefore the limb port assembly 1300) onto a patient. In some embodiments, the flap portion 1348 may extend from the patient's skin to be accessed and/or attached to the exterior of the patient's skin. In some embodiments, the suture flap 1318 may be used to distinguish the venous port 1300 from the arterial port 1200.

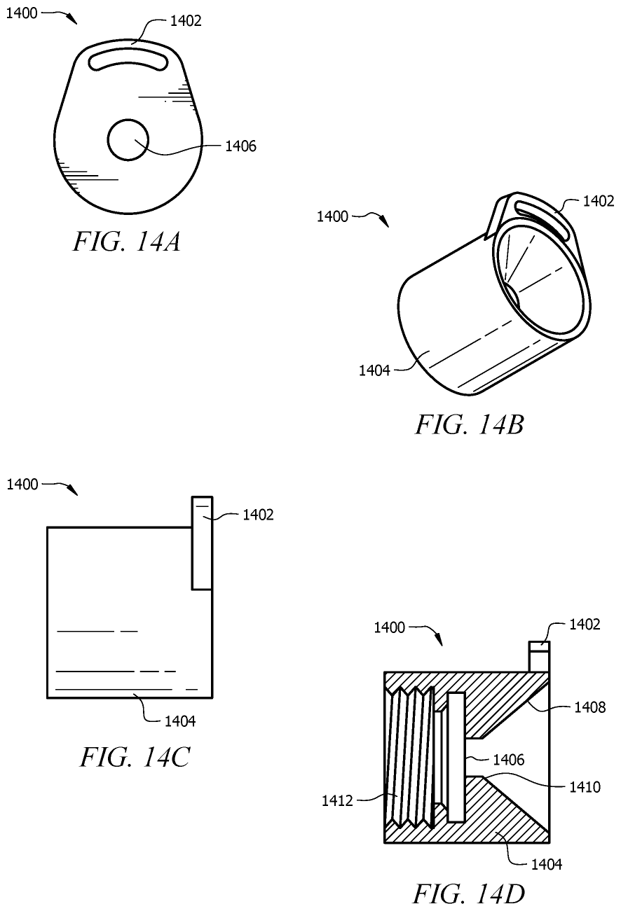

FIGS. 14A-14D illustrate an exemplary embodiment of a needle guide 1400, which may be similar to needle guides 312, 1212, and 1312 described above. The needle guide 1400 may comprise a flange 1402 (and/or lip), an internally tapered body 1404, and a needle aperture 1406. The needle guide 1400 may comprise internal threads 1412 to mate with the threads of the fitting. The tapered body 1404 tapers in its interior from a wide aperture 1408 to a narrow aperture 1410 and then to a needle aperture 1406.

Having described various devices and methods herein, exemplary embodiments or aspects can include, but are not limited to:

In a first embodiment, a dialysis catheter may comprise a lumen tube comprising an inflow lumen and an outflow lumen; a clampless first limb leading to the inflow lumen; a clampless second limb leading to the outflow lumen; a plurality of limb ports attached to the first limb and the second limb, the limb ports comprising: a fitting configured to attach to the first or second limb; a needle guide configured to attach to the fitting, and configured to guide a needle into the first or second limb; and at least one valve positioned between the needle guide and the fitting, configured to prevent fluid flow in one direction.

A second embodiment can include the dialysis catheter of the first embodiment, further comprising a cover for the needle guide configured to seal around a needle when it is inserted into the cover.

A third embodiment can include the dialysis catheter of the first or second embodiments, wherein the fitting comprises threads to mate with threads of the needle guide.

A fourth embodiment can include the dialysis catheter of any of the first through third embodiments, wherein the fitting comprises a first chamber to receive a duckbill of the valve and an outer edge to mate with a flange of the valve.

A fifth embodiment can include the dialysis catheter of the fourth embodiment, wherein the fitting further comprises a second chamber to mate with a lumen of the lumen tube and a transition region between the first chamber and the second chamber.

A sixth embodiment can include the dialysis catheter of any of the first through fifth embodiments, wherein the limb port attached to the first limb leading to the inflow lumen comprises a venous limb port.

A seventh embodiment can include the dialysis catheter of the any of the first through sixth embodiments, wherein the limb port attached to the second limb leading to the outflow lumen comprises an arterial limb port.

An eighth embodiment can include the dialysis catheter of any of the first through seventh embodiments, wherein the limb port comprises a plurality of valves positioned within the limb port.

In a ninth embodiment, a method of inserting a dialysis catheter into a patient may comprise inserting a trocar in the patient in a first direction at an incision site; using the trocar to create a subcutaneous tissue tunnel from the incision site to an exit site from the patient; inserting the trocar at the exit site in a second direction different from the first direction; using the trocar to create a tissue tunnel extension without exiting the patient again; inserting the dialysis catheter into the exit site; extending the dialysis catheter from the exit site through a central vein or jugular vein into a superior vena cava; configuring two limbs of the dialysis catheter each with a limb port housing a valve, a needle guide coupled to the valve, and a cover on the needle guide; and inserting the limbs of the dialysis catheter into the subcutaneous tissue tunnel and the tissue tunnel extension.

A tenth embodiment can include the method of the ninth embodiment, further comprising extending the dialysis catheter over a guidewire from the exit site into the superior vena cava.

An eleventh embodiment can include the method of the tenth embodiment, further comprising deploying a peel-away sheath over the guidewire; removing the guidewire; and extending the dialysis catheter through the exit site into the peel-away sheath and into the superior vena cava while peeling the peel-away sheath.

A twelfth embodiment can include the method of any of the ninth through eleventh embodiments, the needle guide comprising an interior cavity tapered from a wide aperture to a narrow aperture at the interface to the valve.

A thirteenth embodiment can include the method of any of the ninth through twelfth embodiments, wherein the valve is a duckbill valve.

A fourteenth embodiment can include the method of the thirteenth embodiment, wherein the duckbill valve is retained freely between the needle guide and the limb port.

A fifteenth embodiment can include the method of any of the ninth through fourteenth embodiments, wherein each limb is clampless.

In a sixteenth embodiment, a dialysis catheter may comprise a lumen tube bifurcating into a first limb and a second limb; a plurality of limb ports attached to the first limb and the second limb, the limb ports comprising a fitting configured to attach to the first or second limb, and configured to house a valve; and a needle guide configured to attach to the fitting, and configured to retain the valve intermedially between the needle guide and the limb port.

A seventeenth embodiment can include the dialysis catheter of the sixteenth embodiment, the needle guide comprising an interior cavity tapered from a wide aperture to a narrow aperture at the interface to the valve.

An eighteenth embodiment can include the dialysis catheter of the sixteenth or seventeenth embodiments, wherein the valve is a duckbill valve.

A nineteenth embodiment can include the dialysis catheter of the eighteenth embodiment, wherein the duckbill valve is retained freely between the needle guide and the limb port.

A twentieth embodiment can include the dialysis catheter of any of the sixteenth through nineteenth embodiments, wherein the first limb is clampless, and the second limb is clampless.

What is claimed is:

1. A dialysis catheter comprising:
a lumen tube comprising an inflow lumen and an outflow lumen;
a clampless first limb leading to the inflow lumen;
a clampless second limb leading to the outflow lumen;
a plurality of limb ports attached to the first limb and the second limb, the limb ports comprising:
a fitting configured to attach to the first or second limb;
a needle guide configured to attach to the fitting, and configured to guide a needle into the first or second limb; and
at least one valve positioned between the needle guide and the fitting, configured to prevent fluid flow in one direction.

2. The dialysis catheter of claim 1, further comprising a cover for the needle guide configured to seal around a needle when it is inserted into the cover.

3. The dialysis catheter of claim 1, wherein the fitting comprises threads to mate with threads of the needle guide.

4. The dialysis catheter of claim 1, wherein the fitting comprises a first chamber to receive a duckbill of the valve and an outer edge to mate with a flange of the valve.

5. The dialysis catheter of claim 4, wherein the fitting further comprises a second chamber to mate with a lumen of the lumen tube and a transition region between the first chamber and the second chamber.

6. The dialysis catheter of claim 1, wherein the limb port attached to the first limb leading to the inflow lumen comprises a venous limb port.

7. The dialysis catheter of claim 1, wherein the limb port attached to the second limb leading to the outflow lumen comprises an arterial limb port.

8. The dialysis catheter of claim 1, wherein the limb port comprises a plurality of valves positioned within the limb port.

9. A method of inserting a dialysis catheter into a patient, the method comprising:
inserting a trocar in the patient in a first direction at an incision site;
using the trocar to create a subcutaneous tissue tunnel from the incision site to an exit site from the patient;
inserting the trocar at the exit site in a second direction different from the first direction;
using the trocar to create a tissue tunnel extension without exiting the patient again;
inserting the dialysis catheter into the exit site;
extending the dialysis catheter from the exit site through a central vein or jugular vein into a superior vena cava;
configuring two limbs of the dialysis catheter each with a limb port housing a valve, a needle guide coupled to the valve, and a cover on the needle guide; and
inserting the limbs of the dialysis catheter into the subcutaneous tissue tunnel and the tissue tunnel extension.

10. The method of claim 9, further comprising extending the dialysis catheter over a guidewire from the exit site into the superior vena cava.

11. The method of claim 10, further comprising:
deploying a peel-away sheath over the guidewire;
removing the guidewire; and
extending the dialysis catheter through the exit site into the peel-away sheath and into the superior vena cava while peeling the peel-away sheath.

12. The method of claim 9, the needle guide comprising an interior cavity tapered from a wide aperture to a narrow aperture at the interface to the valve.

13. The method of claim 9, wherein the valve is a duckbill valve.

14. The method of claim 13, wherein the duckbill valve is retained freely between the needle guide and the limb port.

15. The method of claim 9, wherein each limb is clampless.

16. A dialysis catheter comprising:
a lumen tube bifurcating into a first limb and a second limb;
a plurality of limb ports attached to the first limb and the second limb, the limb ports comprising:
a fitting configured to attach to the first or second limb, and configured to house a valve; and
a needle guide configured to attach to the fitting, and configured to retain the valve intermedially between the needle guide and the limb port.

17. The dialysis catheter of claim 16, the needle guide comprising an interior cavity tapered from a wide aperture to a narrow aperture at the interface to the valve.

18. The dialysis catheter of claim 16, wherein the valve is a duckbill valve.

19. The dialysis catheter of claim 18, wherein the duckbill valve is retained freely between the needle guide and the limb port.

20. The dialysis catheter of claim 18, wherein the first limb is clampless, and the second limb is clampless.
